(19) United States
(12) Patent Application Publication
(10) Pub. No.: US 2005/0176948 A1
(43) Pub. Date: Aug. 11, 2005

(54) Building block capable of functional entity transfer to nucleophil

(76) Inventors: Alex Haahr Gouliaev, Veksoe Sjaelland (DK); Henrik Pedersen, Bagsvaerd (DK); Thomas Thisted, Frederikssund (DK); Mikkel Dybro Lundorf, Copenhagen NV (DJ); Christian Sams, Frederiksberg C (DK); Thomas Franch, Copenhagen N (DK); Gitte Nystrup Husemoen, Copenhagen N (DK); Justin Ho, Copenhagen V (DK)

Correspondence Address: BROWDY AND NEIMARK, P.L.L.C.;624 NINTH STREET, NW, SUITE 300, WASHINGTON, DC, 20001-5303, US

(21) Appl. No.: 10/507,936

(22) PCT Filed: Mar. 14, 2003
(86) PCT No.: PCT/DK03/00177

(30) Foreign Application Priority Data
Mar. 15, 2002 (DK) PA 2002 0415
Mar. 15, 2002 (US) 60364056
Jun. 20, 2002 (DK) PCTDK 02/00419
Jun. 20, 2002 (US) 10175539
Dec. 19, 2002 (US) 60434439

(51) Int. Cl. C07H 21/00

(52) U.S. Cl. 536/25.32; 536/23.1; 435/6

(57) ABSTRACT

A building block having the dual capabilities of transferring the genetic information e.g. by recognising an encoding element and transferring a functional entity to a recipient reactive group is diclosed. The building block can be designed with an adjustable transferability taking into account the components of the building block. The building block may be used in the generation of a single complex or libraries of different complexes, wherein the complex comprises an encoded molecule linked to an encoding element. Libraries of complexes are useful in the quest for pharmaceutically active compounds.

### TECHNICAL FIELD OF THE INVENTION

The present invention relates to a building block comprising a complementing element and precursor for a functional entity. The building block is designed to transfer the functional entity with an adjustable efficiency to a recipient reactive group upon recognition between the complementing element and an encoding element associated with the reactive group. The invention also relates to a linkage between the functional entity and the complementing element as well as a method for transferring a functional entity to recipient reactive group.

### BACKGROUND

The transfer of a chemical entity from one mono-, di- or oligonucleotide to another has been considered in the prior art. Thus, N. M. Chung et al. (Biochim. Biophys. Acta, 1971, 228,536-543) used a poly(U) template to catalyse the transfer of an acetyl group from 3′-O-acetyladenosine to the 5′-OH of adenosine. The reverse transfer, i.e. the transfer of the acetyl group from a 5′-O-acetyladenosine to a 3′-OH group of another adenosine, was also demonstrated.

Waider et al. Proc. Natl. Acad. Sci. USA, 1979, 76, 51-55 suggest a synthetic procedure for peptide synthesis. The synthesis involves the transfer of nascent immobilized polypeptide attached to an oligonucleotide strand to a precursor amino acid attached to an oligonucleotide. The transfer comprises the chemical attack of the amino group of the amino acid precursor on the substitution labile peptidyl ester, which in turn results in an acyl transfer. It is suggested to attach the amino acid precursor to the 5′ end of an oligonucleotide with a thiol ester linkage.

The transfer of a peptide from one oligonucleotide to another using a template is disclosed in Bruick R K et al. Chemistry & Biology, 1996, 3:49-56. The carboxy terminal of the peptide is initially converted to a thioester group and subsequently transformed to an activated thioester upon incubation with Ellman's reagent. The activated thioester is reacted with a first oligo, which is 5′-thiol-terminated, resulting in the formation of a thio-ester linked intermediate. The first oligonucleotide and a second oligonucleotide having a 3′ amino group is aligned on a template such that the thioester group and the amino group are positioned in close proximity and a reaction is effected resulting in a coupling of the peptide to the second oligonucleotide through an amide bond.

The prior art building blocks and methods for transfer have a relatively poor transfer efficiency. Therefore, in an aspect of the present invention an oligonucleotide conjugated to a transferable chemical moiety via a linker is provided, which has an increased ability to transfer a functional entity.

### SUMMARY OF THE INVENTION

The present invention relates to a building block of the general formula 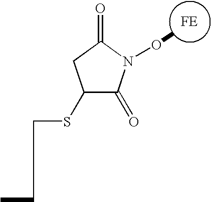
capable of transferring a functional entity (FE) to a recipient reactive group, wherein

the lower horizontal line is a Complementing Element identifying the functional entity and the vertical line between the complementing element and the S atom is a Spacer.

Preferably the spacer is a valence bond, C₁-C₆ alkylene-A-, C₁-C₆ alkenylene-A-, C₂-C₆ alkynylene-A-, or 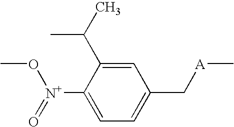
said spacer optionally being connected through A to a moiety selected from 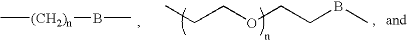
—(CH₂)ₙ—S—S—(CH₂)ₘ—B—
where A is a valence bond, —C(O)NR¹—, —NR¹—, —O—, —S—, or —C(O)—O—; B is a valence bond, —O—, —S—, —NR¹— or —C(O)NR¹— and connects to the S atom of the carrier; R¹ is selected independently from H, C₁-C₆alkyl, C₃-C₇ cycloalkyl, C₁-C₆ alkylene-aryl, or aryl substituted with 0-5 halogen atoms selected from —F, —Cl, —Br and —I; and n and m independently are integers ranging from 1 to 10.

In one aspect of the invention the Spacer is C₁-C₆ alkylene-A-, C₁-C₆ alkenylene-A-, C₂-C₆ alkynylene-A-, or 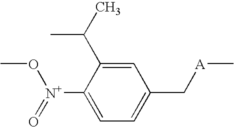
said spacer optionally being connected through A to a moiety selected from 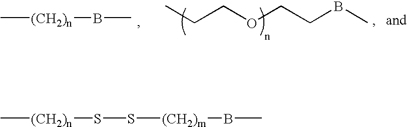
where A is —C(O)NR¹—, or —S—; B is —S—, —NR¹— or —C(O)NR¹— and connects to S—C-connecting group; R¹ is selected independently from H, C₁-C₆ alkyl, C₁-C₆ alkylene-aryl, or aryl; and n and m independently are integers ranging from 1 to 6.

Preferably the Spacer is -A-, a group C₁-C₆ alkylene-A-, C₂-C₆ alkenylene-A-, or C₂-C₆ alkynylene-A- optionally substituted with 1 to 3 hydroxy groups, or 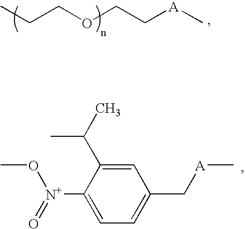
said spacer being connected through A to a linker selected from 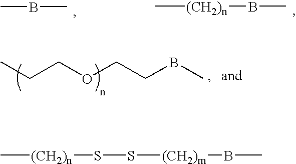
where A is a valence bond, —NR²—, —C(O)NR²—, —NR²—C(O)—, —O—, —S—, —C(O)—O— or OP(═O)(O⁻)—O—; B is a valence bond, —O—, —S—, —NR²—, —C(O)— or —C(O)NR²— and connects to S-C-connecting group; R² is selected independently from H, C₁-C₆ alkyl, C₃-C₇ cycloalkyl, aryl, C₁-C₆ alkylene-aryl, 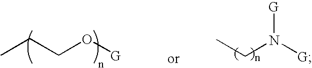
G is H or C₁-C₆ alkyl; and n and m independently are integers ranging from 1 to 10.

The spacer may connect to the complementing element in any convenient way. When the complementing element is a nucleic acid, the spacer may connect to the backbone or the nucleobase. In one aspect of the invention, the spacer is C₂-C₆ alkenylene-A,
said spacer being connected through A to a moiety selected from 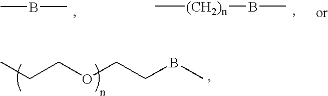
where A is a valence bond, —C(O)NR²—, —NR²—C(O)—, —S—, —C(O)—O— or OP(═O)(O⁻)—O—; B is a valence bond, —S—, —NR²—, or —C(O)— and connects to S-C-connecting group; n and m independently are integers ranging from 1 to 10 and R² is selected independently from H, 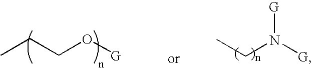
wherein G is H or C₁-C₆ alkyl; and the spacer is connected to the complementing element through a nucleobase.

Suitably, the spacer is attached to the 5 position of a pyrimidine type nucleobase or 7 position of a purine or 7deaza-purine type nucleobase. However, other position of attachment may be appropriate.

In another aspect of the invention the spacer is -A-, 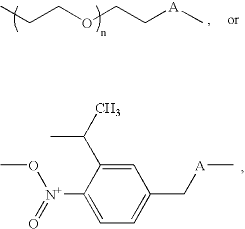
said spacer being connected through A to a moiety selected from 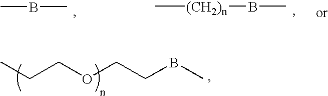
where A is a valence bond, —NR²—C(O)—, —O—, or —S—; B is a valence bond, —S—, —NR²—, or —C(O)— and connects to S-C-connecting group;

n and m independently are integers ranging from 1 to 10 and

R² is selected independently from H, 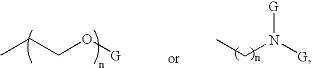
wherein G is H or C₁-C₆ alkyl; and the spacer is connected to the complementing element via a phosphorus group.

The phosphorus group is suitable a phosphate or thiophosphate group attached to a 3′ or 5′ end of a complementing element.

The building block according to the present invention can transfer a variety of chemical compounds to a recipient reactive group. In one aspect of the invention the functional entity is of the format, 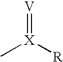
where X=—C—, —S—, —P—, —S(O)—, —P(O)—, and V=O, S, NH, N-C₁-C₆ alkyl. R may be chosen from any chemical group capable of forming a chemical bond to the X atom. In a preferred aspect of the invention FE is 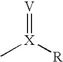
where

X=—C—, —S—, —P—, —S(O)—, or —P(O)—, V=O, S, NH, or N-C₁-C₆ alkyl, and

R is H or selected among the group consisting of a C₁-C₆ alkyl, C₂-C₆ alkenyl, C₂-C₆ alkynyl, C₄-C₈ alkadienyl, C₃-C₇ cycloalkyl, C₃-C₇ cycloheteroalkyl, aryl, and heteroaryl, said group being substituted with 0-3 R⁴, 0-3 R⁵ and 0-3 R⁹ or C₁-C₃ alkylene-NR⁴₂, C₁-C₃ alkylene-NR⁴C(O)R⁸, C₁-C₃alkylene-NR⁴C(O)OR⁸, C₁-C₂ alkylene-O-NR⁴₂, C₁-C₂ alkylene-O-NR⁴C(O)R¹⁸, C₁-C_{2 l alkylene-O-NR}⁴C(O)OR⁸ substituted with 0-3 R⁹.

where R⁴ is H or selected independently among the group consisting of C₁-C₆ alkyl, C₂-C₆ alkenyl, C₂-C₆ alkynyl, C₃-C₇ cycloalkyl, C₃-C₇ cycloheteroalkyl, aryl, heteroaryl, said group being substituted with 0-3 R⁹ and

R⁵ is selected independently from —N₃, —CNO, —C(NOH)NH₂, —NHOH, —NHNHR⁶, —C(O)R⁶, —SnR⁶₃, —B(OR⁶)₂, —P(O)(OR⁶)₂ or the group consisting of C₂-C₆ alkenyl, C₂-C₆ alkynyl, C₄-C₈ alkadienyl said group being substituted with 0-2 R⁷,

where R⁶ is selected independently from H, C₁-C₆ alkyl, C₃-C₇ cycloalkyl, aryl or C₁-C₆ alkylene-aryl substituted with 0-5 halogen atoms selected from —F, —Cl, —Br, and —I; and R⁷ is independently selected from —NO₂, —COOR⁶, —COR⁶, —CN, —OSiR⁶₃, —OR⁶ and —NR⁶₂.

R⁸ is H, C₁-C₆ alkyl, C₂-C₆ alkenyl, C₂-C₆ alkynyl, C₃-C₇ cycloalkyl, aryl or C₁-C₆ alkylene-aryl substituted with 0-3 substituents independently selected from —F, —Cl, —NO₂, —R³, —OR³, —SiR³₃

R⁹ is ═O, —F, —Cl, —Br, —I, —CN, —NO₂, —OR⁶, —NR⁶₂, —NR⁶—C(O)R⁶, —NR⁶—C(O)OR⁸, —SR⁶, —S(O)R⁸, —S(O)₂R⁶, —COOR⁶, —C(O)NR⁶₂ and —S(O)₂NR⁶₂.

In a certain aspect of the invention, R is H or selected among the group consisting of a C₁-C₆ alkyl, C₂-C₆ alkenyl, C₂C₆ alkynyl, C₄-C₆ alkadienyl, C₃-C₇ cycloalkyl, C₃-C₇ cycloheteroalkyl, aryl, and heteroaryl, said group being substituted with 0-3 R⁵ and 0-3 R⁹, or selected among the group consisting of C₁-C₃ alkylene-NR⁴₂, C₁-C₃ alkylene-NR⁴C(O)R⁸, C₁-C₃ alkylene-NR⁴C(O)OR⁸, C₁-C₂ alkylene-O-NR⁴₂, C₁-C₂ alkylene-ONR⁴C(O)R⁸, and C₁-C₂alkylene-O-NR⁴C(O)OR⁸ substituted with 0-3 R⁹.

Suitably, R is H or selected among the group consisting of C₁-C₆ alkyl, C₂-C₆ alkenyl, C₂-C₆ alkynyl, C₄-C₈ alkadienyl, C₃-C₇ cycloalkyl, C₃-C₇ cycloheteroalkyl, aryl, and heteroaryl, said group being substituted with 0-3 R⁵ and 0-3 R⁹.

In some aspects of the invention it is preferred that R is selected among the group consisting of C₁-C₃ alkylene-NR⁴₂, C₁-C₃ alkylene-NR⁴C(O)R⁸, C₁-C₃ alkylene-NR⁴C(O)OR⁸, C₁-C₂alkylene-O—NR⁴₂, C₁-C₂alkylene-O—NR⁴C(O)R⁸, and C₁-C₂ alkylene-O—NR⁴C(O)OR⁸ substituted with 0-3 R⁹.

In the present description and claims, the direction of connections between the various components of a building block should be read left to right. For example a spacer is connected to a complementing element through the atom on the left and to the sulphur atom (or alternatively the group A) through the atom on the right hand side.

The term “C₃-C₇ cycloheteroalkyl” as used herein refers to a radical of totally saturated heterocycle like a cyclic hydrocarbon containing one or more heteroatoms selected from nitrogen, oxygen, phosphor, boron and sulphur independently in the cycle such as pyrrolidine (1-pyrrolidine; 2-pyrrolidine; 3-pyrrolidine; 4-pyrrolidine; 5-pyrrolidine); pyrazolidine (1-pyrazolidine; 2-pyrazolidine; 3-pyrazolidine; 4-pyrazolidine; 5-pyrazolidine); imidazolidine (1- imidazolidine; 2-imida-zolidine; 3-imidazolidine; 4-imidazolidine; 5-imidazolidine); thiazolidine (2-thiazolidine; 3-thiazolidine; 4-thiazolidine; 5-thiazolidine); piperidine (1-piperidine; 2-piperidine; 3-piperidine; 4-piperidine; 5-piperidine; 6-piperidine); piperazine (1-piperazine; 2-piperazine; 3-piperazine; 4-piperazine; 5-piperazine; 6-piperazine); morpholine (2-morpholine; 3-morpholine; 4-morpholine; 5-morpholine; 6-morpholine); thiomorpholine (2-thiomorpholine; 3-thiomorpholine; 4-thiomorpholine; 5-thiomorpholine; 6-thiomorpholine); 1,2-oxathiolane (3-(1,2-oxathiolane); 4-(1,2-oxathiolane); 5-(1,2-oxathiolane); 1,3-dioxolane (2-(1,3-dioxolane); 4-(1,3-dioxolane); 5-(1,3-dioxolane); tetrahydropyrane; (2-tetrahydropyrane; 3-tetrahydropyrane; 4-tetrahydropyrane; 5-tetrahydropyrane; 6-tetrahydropyrane); hexahydropyridazine (1-(hexahydropyridazine); 2-(hexahydropyridazine); 3-(hexahydropyridazine); 4-(hexahydropyridazine); 5-(hexahydropyridazine); 6-(hexahydropyridazine)), [1,3,2]dioxaborolane, [1,3,6,2]dioxazaborocane

The term “aryl” as used herein includes carbocyclic aromatic ring systems of 5-7 carbon atoms. Aryl is also intended to include the partially hydrogenated derivatives of the carbocyclic systems as well as up to four fused aromatic- or partially hydrogenated rings, each ring comprising 5-7 carbon atoms.

The term “heteroaryl” as used herein includes heterocyclic unsaturated ring systems containing, in addition to 2-18 carbon atoms, one or more heteroatoms selected from nitrogen, oxygen and sulphur such as furyl, thienyl, pyrrolyl, heteroaryl is also intended to include the partially hydrogenated derivatives of the heterocyclic systems enumerated below.

The terms “aryl” and “heteroaryl” as used herein refers to an aryl which can be optionally substituted or a heteroaryl which can be optionally substituted and includes phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-5-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine4-yl, 10,11-dihydro-5H-dibenz[b,f]azepine-5-yl).

The Functional Entity carries elements used to interact with host molecules and optionally reactive elements allowing further elaboration of an encoded molecule of a library. Interaction with host molecules like enzymes, receptors and polymers is typically mediated through van der waal's interactions, polar- and ionic interactions and pi-stacking effects. Substituents mediating said effects may be masked by methods known to an individual skilled in the art (Greene, T. W.; Wuts, P. G. M. Protective Groups in Organic Synthesis; 3rd ed.; John Wiley & Sons: New York, 1999.) to avoid undesired interactions or reactions during the preparation of the individual building blocks and during library synthesis. Analogously, reactive elements may be masked by suitably selected protection groups. It is appreciated by one skilled in the art that by suitable protection, a functional entity may carry a wide range of substituents.

The Functional Entity may be a masked Functional Entity that is incorporated into an encoded molecule. After incorporation, reactive elements of the Functional Entity may be revealed by unmasking allowing further synthetic operations. Finally, elements mediating recognition of host molecules may be un-masked.

The function of the carrier 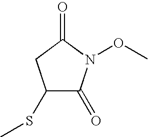
is to provide for the transferability of the functional entity, playing the role of a leaving group.

The spacer serves to distance the functional entity to be transferred from the bulky complementing element. Thus, the identity of the spacer is not crucial for the function of the building block. It may be desired to have a spacer which can be cleaved by light. In this occasion, the spacer is provided with e.g. the group

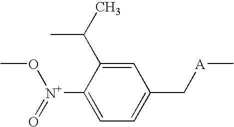

In the event an increased hydophilicity is desired the spacer may be provided with a polyethylene glycol part of the general formula:

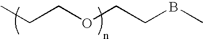

The spacer in conjunction with the carrier makes up a cleavable linker, which links the complementing element to the functional entity.

In a preferred embodiment, the complementing element serves the function of transferring genetic information e.g. by recognising a coding element. The recognition implies that the two parts are capable of interacting in order to assemble a complementing element—coding element complex. In the biotechnological field a variety of interacting molecular parts are known which can be used according to the invention. Examples include, but are not restricted to protein-protein interactions, protein-polysaccharide interactions, RNA-protein interactions, DNA-DNA interactions, DNA-RNA interactions, RNA-RNA interactions, biotin-streptavidin interactions, enzyme-ligand interactions, antibody-ligand interaction, protein-ligand interaction, ect.

The interaction between the complementing element and coding element may result in a strong or a week bonding. If a covalent bond is formed between the parties of the affinity pair the binding between the parts can be regarded as strong, whereas the establishment of hydrogen bondings, interactions between hydrophobic domains, and metal chelation in general results in weaker bonding. In general relatively weak bonding is preferred. In a preferred aspect of the invention, the complementing element is capable of reversible interacting with the coding element so as to provide for an attachment or detachment of the parts in accordance with the changing conditions of the media.

In a preferred aspect of the invention, the interaction is based on nucleotides, i.e. the complementing element is a nucleic acid. Preferably, the complementing element is a sequence of nucleotides and the coding element is a sequence of nucleotides capable of hybridising to the complementing element. The sequence of nucleotides carries a series of nucleobases on a backbone. The nucleobases may be any chemical entity able to be specifically recognized by a complementing entity. The nucleobases are usually selected from the natural nucleobases (adenine, guanine, uracil, thymine, and cytosine) but also the other nucleobases obeying the Watson-Crick hydrogen-bonding rules may be used, such as the synthetic nucleobases disclosed in U.S. Pat. No. 6,037,120. Examples of natural and non-natural nucleobases able to perform a specific pairing are shown in FIG. 2. The backbone of the sequence of nucleotides may be any backbone able to aggregate the nucleobases is a sequence. Examples of backbones are shown in FIG. 4. In some aspects of the invention the addition of non-specific nucleobases to the complementing element is advantageous, FIG. 3.

The coding element can be an oligonucleotide having nucleobases which complements and is specifically recognised by the complementing element, i.e. in the event the complementing element contains cytosine, the coding element part contains guanine and visa versa, and in the event the complementing element contains thymine or uracil the coding element contains adenine.

The complementing element may be a single nucleobase. In the generation of a library, this will allow for the incorporation of four different functional entities into the template-directed molecule. However, to obtain a higher diversity a complementing element preferably comprises at least two and more preferred at least three nucleotides. Theoretically, this will provide for 4² and 4³, respectively, different functional entities uniquely identified by the complementing element. The complementing element will usually not comprise more than 100 nucleotides. It is preferred to have complementing elements with a sequence of 3 to 30 nucleotides.

The building blocks of the present invention can be used in a method for transferring a functional entity to a recipient reactive group, said method comprising the steps of
providing one or more building blocks as described above and
contacting the one or more building blocks with a corresponding coding element associated with a recipient reactive group under conditions which allow for a recognition between the one or more complementing elements and the coding elements, said contacting being performed prior to, simultaneously with, or subsequent to a transfer of the functional entity to the recipient reactive group.

The coding element may comprise one, two, three or more codons, i.e. sequences that may be specifically recognised by a complementing element. Each of the codons may be separated by a suitable spacer group. Preferably, all or at least a majority of the codons of the template are arranged in sequence and each of the codons are separated from a neighbouring codon by a spacer group. Generally, it is preferred to have more than two codons on the template to allow for the synthesis of more complex encoded molecules. In a preferred aspect of the invention the number of codons of the encoding element is 2 to 100. Still more preferred are coding elements comprising 3 to 10 codons. In another aspect, a codon comprises 1 to 50 nucleotides and the complementing element comprises a sequence of nucleotides complementary to one or more of the encoding sequences.

The recipient reactive group may be associated with the encoding element in any appropriate way. Thus, the reactive group may be associated covalently or non-covalently to the coding element. In one embodiment the recipient reactive group is linked covalently to the encoding element through a suitable linker which may be separately cleavable to release the reaction product. In another embodiment, the reactive group is coupled to a complementing element, which is capable of recognising a sequence of nucleotides on the encoding element, whereby the recipient reactive group becomes attached to the encoding element by hybridisation. Also, the recipient reactive group may be part of a chemical scaffold, i.e. a chemical entity having one or more reactive groups available for receiving a functional entity from a building block.

The recipient reactive group may be any group able to cleave the bond between the carrier and the functional entity to release the functional entity. Usually, the reactive group is nucleophilic, such as a hydroxyl, a thiol, an amine etc. A preferred recipient reactive group is an amine group. The nucleophile usually attacks the atom of the functional entity connected to the oxygen attached to the nitrogen ring member of the carrier. When the functional entity is attached to said oxygen through a group X═V, the nucleophile attacks the X atom, thereby causing the carrier group to be a leaving group of the reaction, transferring the X(═V)-Functional entity precursor to the recipient. The chemical structure formed has, in the event the nucleophilic group is an amine attached to a scaffold, the general formula:
Scaffold-NH—X(═V)—R

In which

X=—C—, —S—, —P—, —S(O)—, —P(O)—, and

V=O, S, NH, N-C₁-C₆ alkyl, and R is as previously defined.

In a preferred aspect X is C and V is O.

The conditions which allow for transfer to occur are dependent upon the receiving reactive group. Below various examples of the conditions for a transfer to occur are depicted together with the reaction products formed.

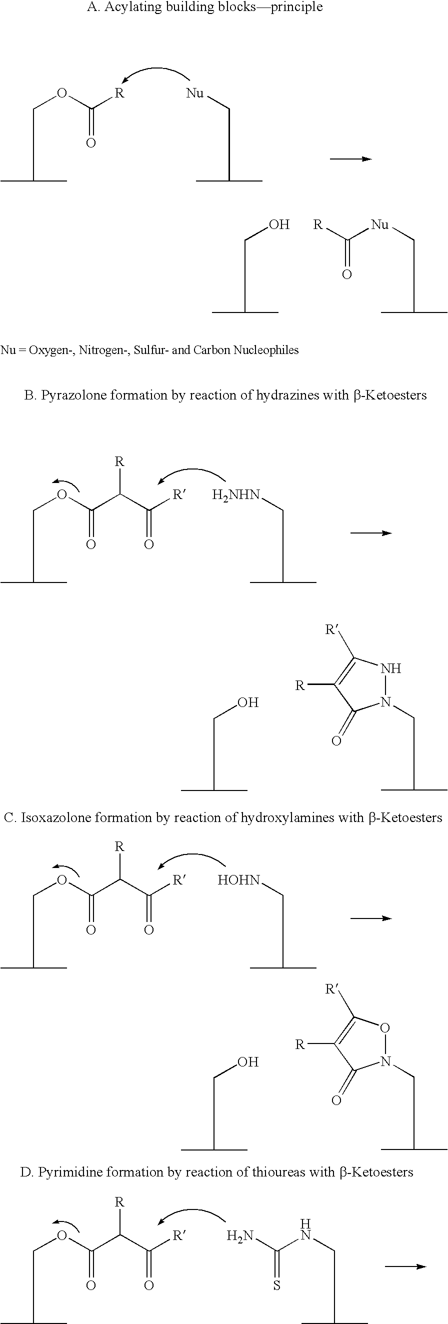

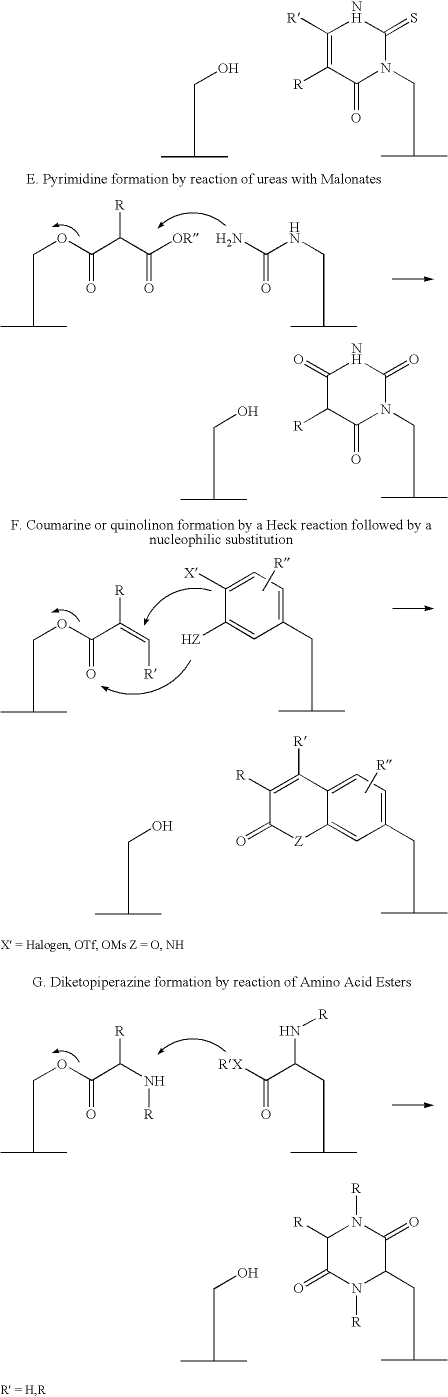

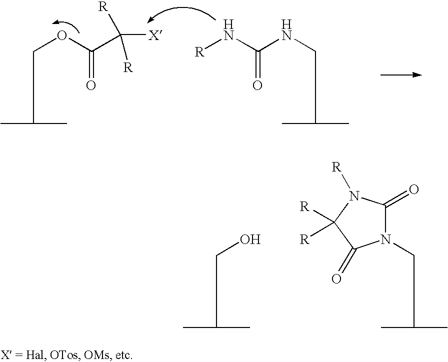

The present building blocks may be prepared in accordance with a variety of chemical synthesis schemes. Generally, a complementing element containing a thiol group is provided. In the event, the complementing element is a oligonucleotide, the thiol may be provided during the synthesis of the oligonucleotide by incorporating a suitable nucleotide derivative. When a oligonucleotide comprising a thiol group is desired, a variety of commercial nucleotide derivatives are available, e.g. the C6 S—S thiol modifier (obtainable from Glen Research cat. # 10-1936-90), which may be incorporated using the standard protocol of the phosphoramedite synthesis.

According to a first synthesis scheme the building block can be prepared using the step

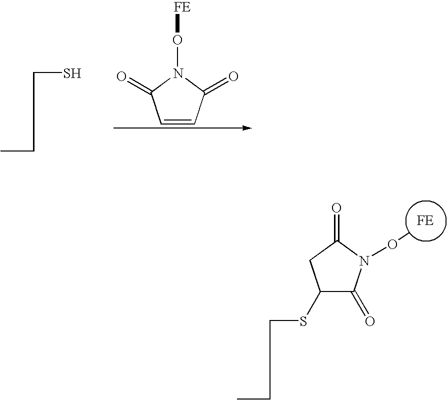

The thiol oligonucleotide is reacted with the N-hydroxymaleimide-functional entity derivative via a Michael addition, whereby the SH group is added to the double bond of the maleimide.

According to a second synthesis scheme, the building blocks can be prepared in two step:

Error! Reference Source Not Found.

The thiol oligonucleotide is reacted with N-hydroxymaleimide via a Michael addition, whereby the SH group is added to the double bond of the maleimide forming an intermediate oligonucleotide derivative which is reacted further with a functional entity connected to a leaving group (Lg). Preferred leaving groups are

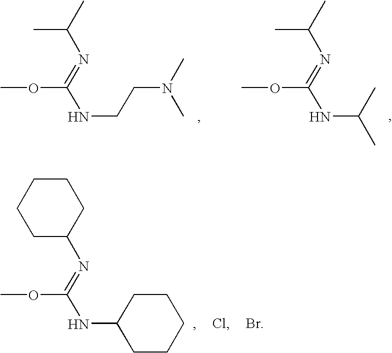

According to a preferred aspect of the invention the building blocks are used for the formation of a library of compounds. The complementing element of the building block is used to identify the functional entity. Due to the enhanced proximity between reactive groups when the complementing entity and the encoding element are contacted, the functional entity together with the identity programmed in the complementing element is transferred to the encoding element associated with recipient reactive group. Thus, it is preferred that the sequence of the complementing element is unique in the sense that the same sequence is not used for another functional entity. The unique identification of the functional entity enable the possibility of decoding the encoding element in order to determine the synthetic history of the molecule formed. In the event two or more functional entities have been transferred to a scaffold, not only the identity of the transferred functional entities can be determined. Also the sequence of reaction and the type of reaction involved can be determined by decoding the encoding element. Thus, according to a preferred embodiment of the invention, each different member of a library comprises a complementing element having a unique sequence of nucleotides, which identifies the functional entity.

### BRIEF DESCRIPTION OF THE DRAWINGS

FIG. 1 shows to setups for functional entity transfer.

FIG. 2 shows examples of specific base pairing

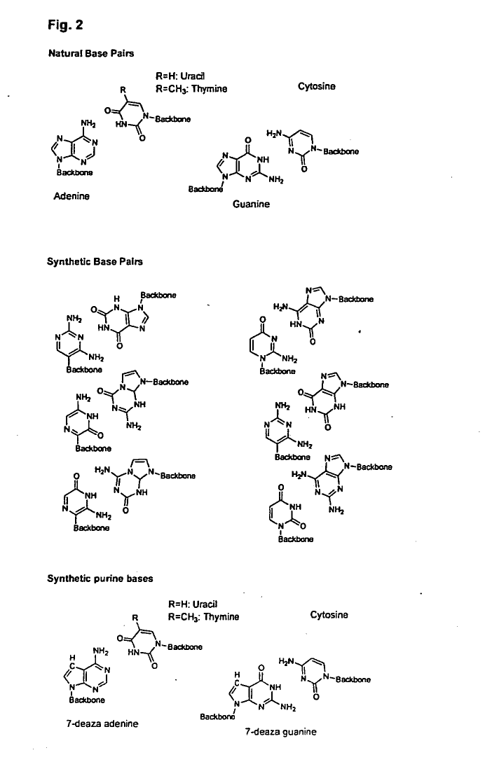

FIG. 3 shows examples of non-specific base-pairing

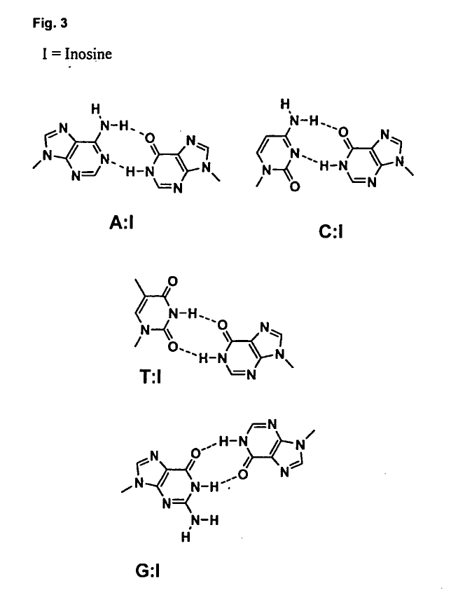

FIG. 4 shows examples of backbones.

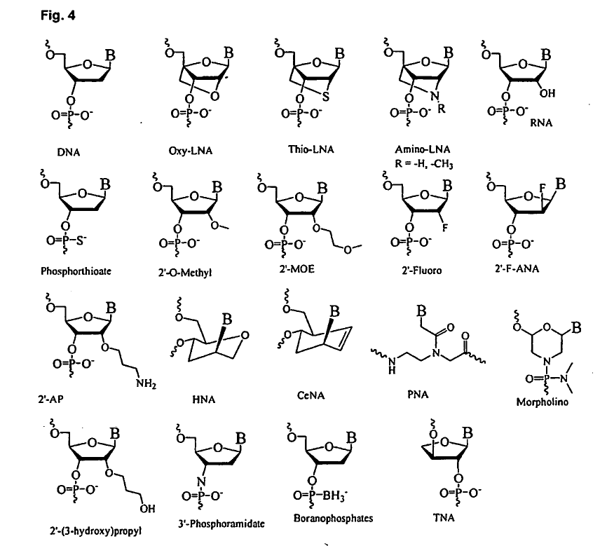

FIG. 5 discloses the results of example 7.

FIG. 6 discloses the results of example 8.

### DETAILED DESCRIPTION OF THE INVENTION

A building block of the present invention is characterized by its ability to transfer its functional entity to a receiving chemical entity. This is done by forming a new covalent bond between the receiving chemical entity and cleaving the bond between the carrier moiety and the functional entity of the building block.

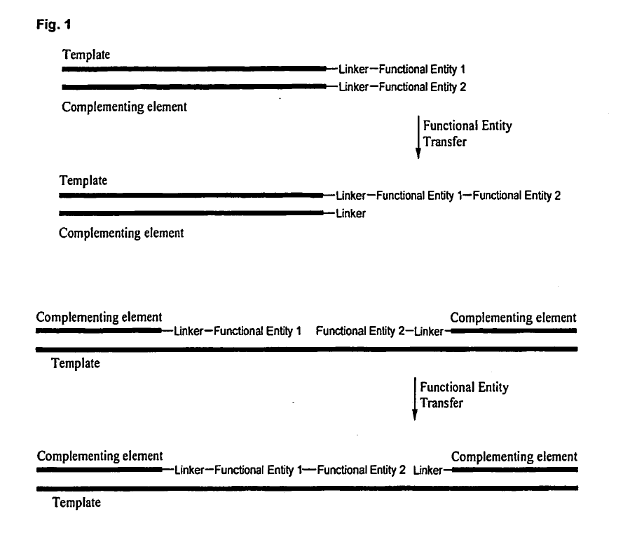

Two setups for generalized functional entity transfer from a building block are depicted in FIG. 1. In the first example, one complementing element of a building block recognizes a template carrying another functional entity, hence bringing the functional entities in close proximity. This results in a reaction between functional entity 1 and 2 forming a covalent bond between these concurrent with the cleavage of the bond between functional entity 2 and its linker. In the second example, a template brings together two building blocks resulting in functional entity transfer from one building block to the other.

In a library synthesis, several building blocks are mixed in a reaction vessel and the added templates ensure that the building blocks—consequently the functional entities—are combined in the desired manner. As several building blocks are employed at the same time, the use of in situ generated building blocks is disfavoured for practical reasons.

Building blocks for library synthesis should posses the necessary reactivity to enable the transfer of the functional entity but should also be stable enough to endure storage and the conditions applied during library synthesis. Hence fine tuning of the reactivity for a particular building block is vital. The reactivity of a building block depends partly on the characteristics of the functional entity and the characteristics of the carrier. E.g. a highly reactive functional entity attached to a highly reactive carrier would form a building block that may be susceptible to hydrolysis during the library synthesis thus preventing successful transfer of one functional entity to another. Further, if transfer of a functional entity precursor is faster than coding element—complementing element recognition unspecific reactions may result.

Therefore, the present invention particularly relates to practically useful library building blocks capable of acting as acylating agents, thioacetylating agents or amidinoylating agents with a balanced reactivity. Such building blocks may be assembled by several different pathways as described below.

The R group of the Functional entity, may be selected from any transferable chemical group capable of forming a connection to —X(═V)— group. In certain aspects of the invention the functional entity precursor is represented by the formula Z²R¹⁷

wherein Z is absent, O, S or NR²⁴. In certain embodiment Z is absent. In a another embodiment Z is O. In still another embodiment Z is S, and in still a further embodiment Z is NR²⁴.

R¹⁷ and R²⁴ independently is H, alkyl, alkenyl, alkynyl, alkadienyl, cycloalkyl, cycloheteroalkyl, aryl or heteroaryl, optionally substituted with one or more substituents selected from the group consisting of SnR¹⁸R¹⁹, R²⁰, Sn(OR¹⁸)R¹⁹R²⁰, Sn(OR¹⁸)(OR¹⁹)R²⁰, BR¹⁸R¹⁹, B(OR¹⁸)R¹⁹, B(OR¹⁸)(OR¹⁹), halogen, CN, CNO, C(halogen)₃, OR¹⁸, OC(═O)R¹⁸, OC(═O)OR¹⁸, OC(═O)NR¹⁸R¹⁹, SR¹⁸, S(═O)R¹⁸, S(═O)₂R¹⁸, S(═O)₂NR¹⁸R¹⁹, NO₂, N₃, NR¹⁸R¹⁹, N⁺R¹⁸R¹⁹R²⁰, NR¹⁸OR¹⁹, NR¹⁸NR¹⁹R²⁰, NR¹⁸C(═O)R¹⁹, NR¹⁸C(═O)OR¹⁹, NR¹⁸C(═O)NR¹⁹R²⁰, NC, P(═O)(OR¹⁸)OR¹⁹, P⁺R¹⁸R¹⁹R²⁰, C(═O)R¹⁸, C(═NR¹⁸)R¹⁹, C(═NOR¹⁸)R¹⁹, C(═NNR¹⁸R¹⁹), C(═O)OR¹⁸, C(═O)NR¹⁸R¹⁹, C(═O)NR¹⁸OR¹⁹, C(═O)NR¹⁸NR¹⁹R²⁰, C(═NR¹⁸)NR¹⁹R²⁰, C(═NOR¹⁸)NR¹⁹R²⁰ or R²¹,

wherein,

R¹⁸, R¹⁹ and R²⁰ independently is H, alkyl, alkenyl, alkynyl, alkadienyl, cycloalkyl, cycloheteroalkyl, aryl or heteroaryl, optionally substituted with one or more substituents selected from the group consisting of halogen, CN, CNO, C(halogen)₃, OR²¹, OC(═O)R²¹, OC(═O)OR²¹, OC(═O)NR²¹R²², SR²¹, S(═O)R²¹, S(═O)₂R²¹, S(═O)₂NR²¹R²², NO₂, N₃, NR²¹R²², N⁺R²¹R²²R²³, NR¹⁸OR¹⁹, NR¹⁸NR¹⁹R²⁰, NR²¹C(═O)R²², NR²¹C(═O)OR²², NR²¹C(═O)NR²²R²³, NC, P(═O)(OR²¹)OR²², P⁺R¹⁸R¹⁹R²⁰, C(═O)R²¹, C(═NR²¹)R²², C(═NOR²¹)R²², C(═NNR²¹R²²), C(═O)OR²¹, C(═O)NR²¹R²², C(═O)NR²¹OR²², C(═NR¹⁸)NR¹⁹R²⁰, C(═NOR¹⁸)NR¹⁹R²⁰ or C(═O)NR²¹NR²²R²³, wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

wherein,

R²¹, R²² and R²³ independently is H, alkyl, alkenyl, alkynyl, alkadienyl, cycloalkyl, cycloheteroalkyl, aryl or heteroaryl and wherein R²¹ and R²² may together form a 3-8 membered heterocyclic ring or R²¹ and R²³ may together form a 3-8 membered heterocyclic ring or R²² and R²³ may together form a 3-8 membered heterocyclic ring,

In a further embodiment,

R¹⁷ and R²⁴ independently is H, C₁-C₆ alkyl, C₂-C₆ alkenyl, C₂-C₆ alkynyl, C₄-C₈ alkadienyl, C₃-C₇ cycloalkyl, C₃-C₇ cycloheteroalkyl, aryl or heteroaryl, optionally substituted with one or more substituents selected from the group consisting of SnR¹⁸R¹⁹,R²⁰, Sn(OR¹⁸)R¹⁹R²⁰, Sn(OR¹⁸)(OR¹⁹)R²⁰, BR¹⁸R¹⁹, B(OR¹⁸)R¹⁹, B(OR¹⁸)(OR¹⁹), halogen, CN, CNO, C(halogen)₃, OR¹⁸, OC(═O)R¹⁸, OC(═O)OR¹⁸, OC(═O)NR¹⁸R¹⁹, SR¹⁸, S(═O)R¹⁸, S(═O)₂R¹⁸, S(═O)₂NR¹⁸R¹⁹, NO₂, N₃, NR¹⁸R¹⁹, N⁺R¹⁸R¹⁹R²⁰, NR¹⁸OR¹⁹, NR¹⁸NR¹⁹R²⁰, NR¹⁸C(═O)R¹⁹, NR¹⁸C(═O)OR¹⁹, NR¹⁸C(═O)NR¹⁹R²⁰, NC, P(═O)(OR¹⁸)OR¹⁹, P⁺R¹⁸R¹⁹R²⁰, C(═O)R¹⁸, C(═NR¹⁸)R¹⁹, C(═NOR¹⁸)R¹⁹, C(═NNR¹⁸R¹⁹), C(═O)OR¹⁸, C(═O)NR¹⁸R¹⁹, C(═O)NR¹⁸OR¹⁹, C(═O)NR¹⁸NR¹⁹R²⁰, C(═NR¹⁸)NR¹⁹R²⁰, C(═NOR¹⁸)NR¹⁹R²⁰ or R²¹,

wherein,

R¹⁸, R¹⁹, R²⁰ and R²¹ independently is H, C₁-C₆ alkyl, C₂-C₆ alkenyl, C₂-C₆ alkynyl, C₄-C₈ alkadienyl, C₃-C₇ cycloalkyl, C₃-C₇ cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 38 membered heterocyclic ring,

In another embodiment,

R¹⁷ and R²⁴ independently is H, C₁-C₆ alkyl, C₃-C₇ cycloalkyl, C₃-C₇ cycloheteroalkyl, aryl or heteroaryl, optionally substituted with one or more substituents selected from the group consisting of halogen, CN, C(halogen)₃, OR¹⁸, OC(═O)R¹⁸, OC(═O)OR¹⁸, OC(═O)NR¹⁸R¹⁹, SR¹⁸, S(═O)R¹⁸, S(═O)₂R¹⁸, S(═O)₂NR¹⁸R¹⁹, NO₂, NR¹⁸R¹⁹, NR¹⁸OR¹⁹, NR¹⁸NR¹⁹R²⁰, NR¹⁸C(═O)R¹⁹, NR¹⁸C(═O)OR¹⁹, NR¹⁸C(═O)NR¹⁹R²⁰, P(═O)(OR¹⁸)OR¹⁹, C(═O)R¹⁸, C(═NR¹⁸)R¹⁹, C(═NOR¹⁸)R¹⁹, C(═NNR¹⁸R¹⁹), C(═O)OR¹⁸, C(═O)NR¹⁸R¹⁹, C(═O)NR¹⁸OR¹⁹, C(═O)NR¹⁸NR¹⁹R²⁰, C(═NR¹⁸)NR¹⁹R²⁰, C(═NOR¹⁸)NR¹⁹R²⁰ or R²¹,

wherein,

R¹⁸, R¹⁹, R²⁰ and R²¹ independently is H, C₁-C₆ alkyl, C₃-C₇ cycloalkyl, C₃-C₇ cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, C₁-C₆ alkyl, C₃-C₇ cycloalkyl, C₃-C₇ cycloheteroalkyl, aryl or heteroaryl, optionally substituted with one or more substituents selected from the group consisting of F, Cl, CN, CF₃, OR¹⁸, OC(═O)R¹⁸, OC(═O)OR¹⁸, OC(═O)NR¹⁸R¹⁹, SR¹⁸, S(═O)R¹⁸, S(═O)₂R¹⁸, S(═O)₂NR¹⁸R¹⁹, NO₂, NR¹⁸R¹⁹, NR¹⁸OR¹⁹, NR¹⁸NR¹⁹R²¹, NR¹⁸C(═O)R¹⁹, NR¹⁸C(═O)OR¹⁹, NR¹⁸C(═O)NR¹⁹R²⁰, P(═O)(OR¹⁸)OR¹⁹, C(═O)R¹⁸, C(═NR¹⁸)R¹⁹, C(═NOR¹⁸)R¹⁹, C(═NNR¹⁸R¹⁹), C(═O)OR¹⁸, C(═O)NR¹⁸R¹⁹, C(═O)NR¹⁸OR¹⁹, C(═O)NR¹⁸NR¹⁹R²⁰, C(═NR¹⁸)NR¹⁹R²⁰, C(═NOR¹⁸)NR¹⁹R²⁰ or R²¹,

wherein,

R¹⁸, R¹⁹, R²⁰ and R²¹ independently is H, C₁-C₆ alkyl, C₃-C₇ cycloalkyl, C₃-C₇ cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, C₁-C₆ alkyl, C₃-C₇ cycloalkyl, C₃-C₇cycloheteroalkyl, aryl or heteroaryl, optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, C₁-C₆ alkyl, C₃-C₇cycloalkyl, C₃-C₇cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, methyl, ethyl, propyl, butyl, cyclopropyl, cyclobutyl, cyclopentyl, cyclohexyl, aziridinyl, azetidinyl, pyrrolidinyl, piperidinyl, morpholinyl, phenyl, naphtyl, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, C₁-C₆ alkyl, C₃-C₇cycloalkyl, C₃-C₇cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, methyl, ethyl, propyl, butyl, cyclopropyl, cyclobutyl, cyclopentyl or cyclohexyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, C₁-C₆ alkyl, C₃-C₇cycloalkyl, C₃-C₇cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, aziridinyl, azetidinyl, pyrrolidinyl, piperidinyl or morpholinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, C₁-C₆ alkyl, C₃-C₇cycloalkyl, C₃-C₇cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, phenyl, naphtyl, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, C₁-C₆ alkyl, C₃-C₇cycloalkyl, C₃-C₇cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 38 membered heterocyclic ring,

In still another embodiment,

R¹⁷ and R²⁴ independently is H, phenyl or naphtyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, C₁-C₆ alkyl, C₃-C₇cycloalkyl, C₃-C₇cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, C₁-C₆ alkyl, C₃-C₇cycloalkyl, C₃-C₇cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, methyl, ethyl, propyl, butyl, cyclopropyl, cyclobutyl, cyclopentyl or cyclohexyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, aziridinyl, azetidinyl, pyrrolidinyl, piperidinyl or morpholinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, phenyl, naphtyl, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, phenyl or naphtyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, methyl, ethyl, propyl, butyl, cyclopropyl, cyclobutyl, cyclopentyl or cyclohexyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁹, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl or butyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, aziridinyl, azetidinyl, pyrrolidinyl, piperidinyl or morpholinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl or butyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, phenyl, naphtyl, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl or butyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, phenyl or naphtyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl or butyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl or butyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is methyl, ethyl, propyl, butyl, cyclopropyl, cyclobutyl, cyclopentyl or cyclohexyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, cyclopropyl, cyclobutyl, cyclopentyl or cyclohexyl.

In still another embodiment,

R¹⁷ and R²⁴ independently is aziridinyl, azetidinyl, pyrrolidinyl, piperidinyl or morpholinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸OR¹⁹, C(═O)NR¹⁸OR¹⁹ or R²¹,

wherein,

R¹⁸, R¹⁹, R²⁰ and R²¹ independently is H, cyclopropyl, cyclobutyl, cyclopentyl or cyclohexyl.

In still another embodiment,

R¹⁷ and R²⁴ independently is phenyl, naphtyl, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, cyclopropyl, cyclobutyl, cyclopentyl or cyclohexyl.

In still another embodiment,

R¹⁷ and R²⁴ independently is phenyl or naphtyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, cyclopropyl, cyclobutyl, cyclopentyl or cyclohexyl.

In still another embodiment,

R¹⁷ and R²⁴ independently is thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁸, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, cyclopropyl, cyclobutyl, cyclopentyl or cyclohexyl,

In still another embodiment,

R¹⁷ and R²⁴ independently is methyl, ethyl, propyl, butyl, cyclopropyl, cyclobutyl, cyclopentyl or cyclohexyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, phenyl, naphthyl, thienyl, furyl, pyridinyl, quinolinyl or isoquinolinyl.

In still another embodiment,

R¹⁷ and R²⁴ independently is aziridinyl, azetidinyl, pyrrolidinyl, piperidinyl or morpholinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, phenyl, naphthyl, thienyl, furyl, pyridinyl, quinolinyl or isoquinolinyl.

In still another embodiment,

R¹⁷ and R²⁴ independently is phenyl, naphtyl, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, phenyl, naphthyl, thienyl, furyl, pyridinyl, quinolinyl or isoquinolinyl.

In still another embodiment,

R¹⁷ and R²⁴ independently is phenyl or naphtyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, phenyl, naphthyl, thienyl, furyl, pyridinyl, quinolinyl or isoquinolinyl.

In still another embodiment,

R¹⁷ and R²⁴ independently is thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, phenyl, naphthyl, thienyl, furyl, pyridinyl, quinolinyl or isoquinolinyl.

In still another embodiment,

R¹⁷ and R²⁴ independently is H, C₁-C₆ alkyl, C₃-C₇ cycloalkyl, C₃-C₇cycloheteroalkyl, aryl or heteroaryl

In still another embodiment,

R¹⁷ and R²⁴ independently is H,

In still another embodiment,

R¹⁷ and R²⁴ independently is C₁-C₆ alkyl, C₃-C₇ cycloalkyl or C₃-C₇cycloheteroalkyl,

In still another embodiment,

R¹⁷ and R²⁴ independently is methyl, ethyl, propyl or butyl

in still another prefered embodiment

R¹⁷ and R²⁴ independently is cyclopropyl, cyclobutyl, cyclopentyl or cyclohexyl

in still another prefered embodiment

R¹⁷ and R²⁴ independently is aziridinyl, pyrrolidinyl, piperidinyl or morpholinyl

In still another embodiment,

R¹⁷ and R²⁴ independently is aryl or heteroaryl

In still another embodiment,

R¹⁷ and R²⁴ independently is phenyl or naphthyl

In still another embodiment,

R¹⁷ and R²⁴ independently is thienyl, furyl, pyridyl, quinolinyl or isoquinolyl

### EXPERIMENTS

All oligos used were prepared by standard phosphoramidite chemistry and purchased from DNA technology, Denmark. The type II compounds used were commercially available from Fluka (4-pentynoic acid cat. no: 77055, 5-hexynoic acid cat. no: 53108 and N-tertbutoxycarbonyl beta-alanin cat. no: 15382). The hexapeptide used as scaffold was synthesised using standard Fmoc chemistry and protected at the N-terminal by acetylation and at the C-terminal by formamide formation. The protected hexapeptide was commercially available from Schaefer-N, Denmark.

### EXAMPLE 1

### Preparation of Type I Compound (Method A)

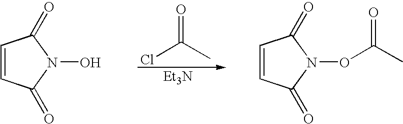

N-hydroxymaleimide (4 mmol) was mixed with Et₃N (4 mmol) in DCM (15 mL) at 0° C. Acetyl chloride (4 mmol) was added and the reaction mixture was left at rt o/n. DCM (15 mL) was added and the reaction mixture was washed with citric acid (3×30 mL), NaHCO₃ (2×30 mL) and NaCl aq. (30 mL). The organic phase was dried over MgSO₄ and evaporated in vacuo to afford acetic acid 2,5-dioxo-2,5-dihydropyrrol-1-yl ester in 41% yield. ¹H NMR (CDCl₃): 6.74 (s, 2H), 2.32 (s, 2H).

### EXAMPLE 2

### Preparation of Building Blocks (Method A)

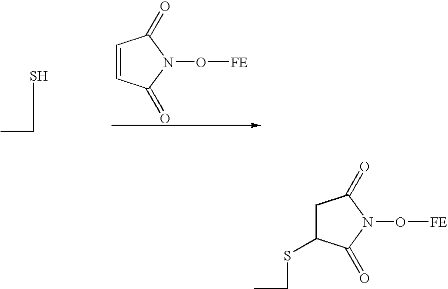

A dTS—S—oligo (10 nmol) is evaporated to dryness in vacuo. The oligo is redissolved in DTT (50 μl 100 mM) in 100 mM Sodium-phosphate buffer pH 8.0. Incubate at 37° C. for 1 h and purify using a micro-spin column equilibrated with Hepes-OH (100 mM, pH 7.5). The HS-oligo is treated with CTAB (50 μL, 1 mM) and the mixture is evaporated to dryness in vacuo. The HS-oligo obtained is redissolved in DMF (100 μL) and treated with compounds of type I (100 μl 100 mM in DMF) for 3 h at rt. NaOAc (200 μl 1 M, pH=7.5) is added and the reaction mixture is extracted with EtOAc (2×300 μL). The loaded oligo is finally purified using a micro-spin column equilibrated with Hepes-OH (100 mM, pH 7.5).

### EXAMPLE 3

### Preparation of Building Blocks (Method B)

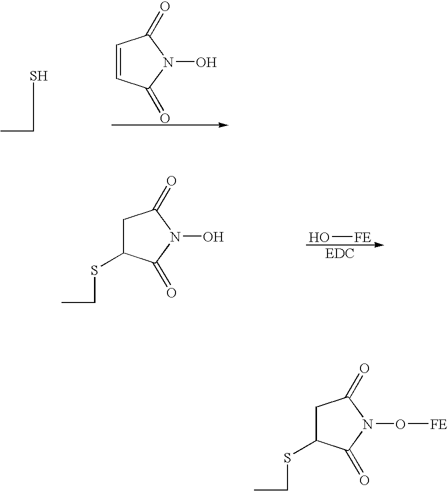

C₆ S-S-oligonucleotides A to D (10 nmol) is evaporated to dryness in vacuo.
A: 5′-GCG ACC TGG AGC ATC CAT CGT S
B: 5′-GAG CAT CCA TCG S
C: 5′-GAC GAG CAT CCA TCG S
D: 5′-CTA GGG ACG AGC ATC CAT CGS
S=Thiol C6 SS modifier (Glen# 10-1936)

The oligo is redissolved in DTT (50 μl 100 mM ) in 100 mM Sodium-phosphate pH 8.0. Incubate at 37° C. for 1 h and purify using a micro-spin column equilibrated with Hepes-OH (100 mM, pH 7.5). NHM (50 μl 100 mM) in HepesOH (100 mM, pH 7.5) is added to the obtained HS-oligo and the mixture is incubated at 25° C. for 2 h. The oligo-S-NHS is then purified using a Microspin columns equilibrated in MS-grade H₂O and analysed by ES-MS.
A: MS (calc): 6723.52; MS (found): 6723.21
B: MS (calc): 3938.75; MS (found): 3937.78
C: MS (calc): 4870.36; MS (found): 4869.42
D: MS (calc): 6435.38; MS (found): 6434.57

Four EDC-activated compounds were prepared by mixing 50 μl 100 mM of each of the compounds (acetic acid, 4-pentynoic acid, N-tertbutoxycarbonyl beta-alanine, and 5-hexynoic acid) in DMF with 50 μl 100 mM of EDC in DMF and leave the mixture at rt for 30 min before use. Subsequently, each of the oligo-S-NHS (1 nmol) is redissolved in MES-buffer (10 μl 100 mM, pH 6) and treated with 10 μl of a DMF solution of the EDC-activated compounds. After 1 h the building blocks are purified using a microspin column equilibrated with 100 mM MES pH6 to obtain
oligonucleotide A loaded with acetyl,
oligonucleotide B loaded with 4-pentynyl (=FE₁),
oligonucleotide C loaded with N-tertbutoxycarbonyl beta-alaninyl (=FE₂), and
oligonucleotide D loaded with 5-hexynyl (FE₃).

ES-MS analysis of the loaded oligonucleotides showed the masses of their corresponding oligo-S-NHS-building blocks shown above, due to the presence of piperidine added during analysis.

### EXAMPLE 4

### Preparation of Scaffold Building Blocks

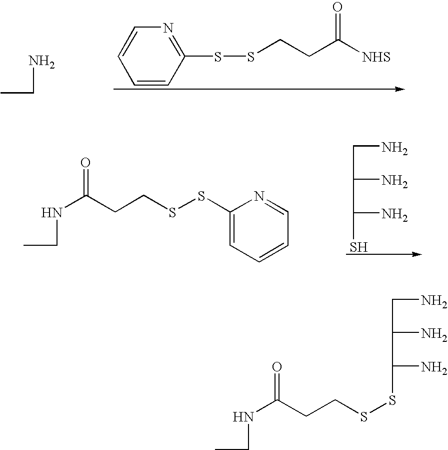

10 nmol of the amino-oligo was diluted in 160 μL 100 mM Hepes-KOH buffer pH 7.5. N-Succinimidyl 3-[2-pyridyldithio]-propionamido, SPDP (40 μl 20 mM, Pierce cat # 21857) was added and the mixture was incubated for 2 h at 30° C. The oligo was extracted with ethyl acetate (200 μL) and purified using micro spin columns equilibrated with 100 mM Hepes-KOH buffer pH 7,5. The hexapeptide CysPhePheLysLysLys (10 μl 100 mM) was added and the mixture was incubated over-night at 30° C. The oligo was purified by ammoniumacetate precipitation and analysed by ES-MS.

MS (calc): 8386.41; MS (found): 8386.57

Used oligo:
E: 5′-X CGA TGG ATG CTC GTC CCT AGA YZ
X=5′-amino modifier C₆ (Glen# 10-1926)
Y=PC spacer (Glen# 104913)
Z=Biotin phosphoramidite (Glen# 10-1955)

### EXAMPLE 5

### Transfer of a Functional Entity

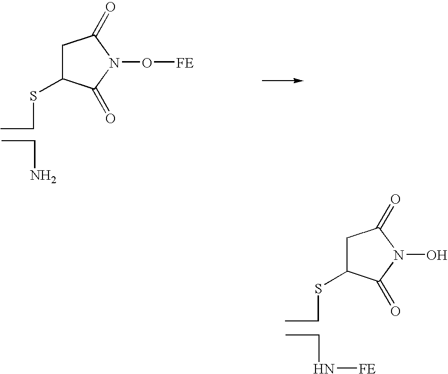

Oligonucleotide A loaded with acetyl (250 pmol) was added to oligo F (200 pmol) in 50 μl 100 mM MES, pH 6. The mixture was incubated overnight at 25° C. Subsequently, the mixture was purified by gel filtration using a microspin column equilibrated with H₂O and transfer of the functional entity was verified by electron spray mass spectrometry (ES-MS).

Used oligos:
A: 5′-GCG ACC TGG AGC ATC CAT CGT-acetyl
F: 5′-X ACG ATG GAT GCT CCA GGT CGC
X=5′ Amino-modifier C₆ (Glen# 10-1906)

MS (calc): 6667.46; MS (found) 6666.64.

### EXAMPLE 6

### Transfer of a Three Different Functional Entities

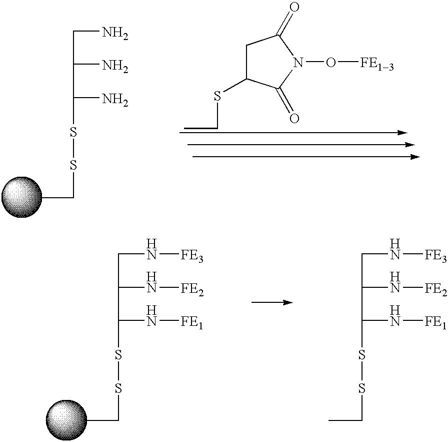

Transfer of the first functional entity: Scaffold building block oligo E (400 pmol) was added to oligo B (400 pmol in 25 μl MES buffer, pH 6), loaded with 4-pentynyl, and incubated over-night at 15° C. The volume was then adjusted to 50 μl and the mixture transferred to a streptavidin-bead slurry (Pharmacia cat #17-5113-01, prewashed with 100 ul MES buffer) and incubated for 10 min at room-temperature, followed by incubation on ice for 10 min. The beads were washed four times with ddH₂O, resuspended in 100 μl 10 mM NaOH and incubated for 2 min at room temperature to denature the duplex. The NaOH was removed and the beads were subsequently washed twice with 60° C. ddH₂O. The water was removed and the beads resuspended in 25 μl 100 mM MES buffer pH 6.0.

Transfer of the second functional entity: Oligo C (400 pmol in 25 μl MES buffer, pH 6), loaded with N-tertbutoxycarbonyl beta-alaninyl, was added to the beads and the mixture was incubated at 25° C. for 2 h. The beads were washed four times with ddH₂O, resuspended in 100 μl 10 mM NaOH and incubated for 2 min at room temperature to denature the duplex. The NaOH was removed and the beads were subsequently washed twice with 60° C. ddH₂O. The water was removed and the beads resuspended in 25 μl 100 mM MES buffer pH 6.0.

Transfer of the third functional entity: Oligo D (400 pmol in 25 μl MES buffer, pH 6), loaded with 5-hexynyl, was added to the beads and the mixture was incubated at 25° C. for 2 h. The beads were washed four times with ddH₂O, resuspended in 100 μl 10 mM NaOH and incubated for 2 min at room temperature to denature the duplex. The NaOH was removed and the beads were subsequently washed twice with 60° C. ddH₂O. The beads were additionally washed once with 50 μl MES buffer and twice with 50 μL water. The beads were resuspended in 25 μl ddH₂O and put on UV transilluminator for 2×15 seconds to cleave oligo E from the beads. 25 μl 12% ammonia was added and the mixture was incubated for 5 min at 50° C. The sample was spun twice at 5 kG, and the supernatant collected. The sample was evaporated to dryness in vacuo, and analysed by ES-MS.
MS of the trisubstituted product (calc): 8197.17
MS of the trisubstituted product (found): 8196.80

[image]

### EXAMPLE 7

### Attachment of Functional Entity to a Thio Oligo

The following oligos containing a nucleobase modified with a S-triphenylmethyl protected thio moiety, were synthesised using the conventional phosphoramidite approach:

L: 5′-WCA TTG ACC TGA ACC ATG BTA AGC TGC CTG TCA
GTC GGT ACT ACG ACT ACG TTC AGG CAA GA
M: 5′-WCA TTG ACC TGA ACC ATG TBA AGC TGC CTG TCA
GTC GGT ACT TCA AGG ATC CAC GTG ACC AG

W was incorporated using the commercially available thiol modifier phosphoramidite (10-1926-90 from Glen research). B is an internal biotin incorporated using the commercially available phosphoramidite (10-1953-95 from Glen research).

To make an SH group available for further reaction, the S-triphenylmethyl protected thio oligo (10 nmol) was evaporated in vacuo and resuspended in TEAA buffer (200 uL of a 0.1 M solution, pH=6.4). AgNO₃ (30 uL of a 1 M solution) was added and the mixture was left at room temperature for 1-2 hours. DTT (46 uL of a 1M solution) was added and left for 5-10 minutes. The reaction mixture was spun down (20.000 G for 20 minutes) and the supernatant was collected. The solid was extracted with additional TEAA buffer (100 ul of a 0.1 M solution, pH=6.4). The pure thio oligo was obtained by conventional EtOH-precipitation.

The L oligo was subsequently reacted with the compound 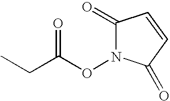
forming a building block able to transfer an acetyl group to a nucleophilic group like an amine, and the M oligo was reacted with the compound 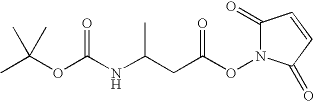
forming a building block capable of transferring a 3-tertbutoxycarbonylamino-butanyl group to a nucleophilic recipient group.

The reaction may be represented by the reaction scheme:

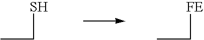

General procedure: The thio oligo (1 nmol) was dried in vacuo and treated with the NHS compound shown above in dimethylformamide (50 ul of a 0.1 M solution) and left o/n at rt. The thio oligo was spun down (20.000 G for 10 minutes) and the supernatant removed. Dimethylformamide (1 mL) was added and the loaded thio oligo was spun down (20.000 G for 10 minutes). The dimethylformamide was removed and the loaded thio oligo was resuspended in TEAA buffer (25 uL of a 0.1M solution, pH=6.4) and analysed by HPLC.

The functional entities were transferred to a amino oligonucleotide according to the scheme:

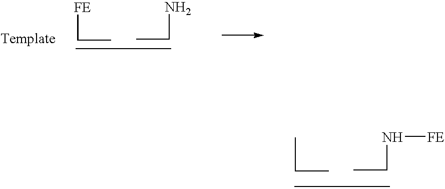

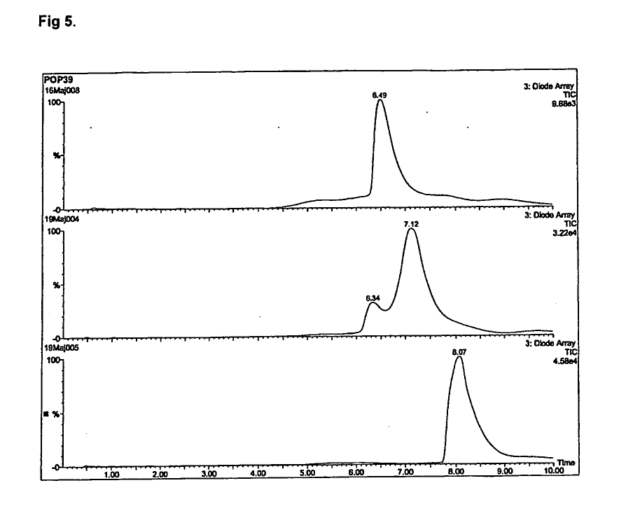

General procedure: The template oligo 5′-BTCTTGCCTGAACGTAGTCGTAGGTCGATCCGCGTTACCAGAGCTGGATGCTC GACAGGTCCCGATGCAATCCAGAGGTCG (1 nmol) was mixed with the oligos (L or M) loaded with a functional entity (1 nmol) and amino oligo O in hepes-buffer (20 uL of a 100 mM HEPES and 1 M NaCl solution, pH=7.5) and water (added to a final volume of 100 uL). The oligos were annealed to the template by heating to 50° C. and cooled (−2° C./30 second) to 30° C. The mixture was then left o/n at a fluctuating temperature (10° C. for 1 second then 35° C. for 1 second). The oligo complex was attached to streptavidine by addition of streptavidine beads (100 uL, prewashed with 2×1 mL 100 mM hepes buffer and 1M NaCl, pH=7.5). The beads were washed with hepes buffer (1 mL). The amino oligo was separated from the streptavidine bound complex by addition of water (200 uL) followed by heating to 70° C. for 1 minute. The water was transferred and evaporated in vacuo, resuspended in TEAA buffer (45 uL of a 0.1 M solution) and product formation analysed by HPLC (see FIG. 5).

FIG. 5 shows the transfer of functional entities to an oligo containing a modified nucleobase with an amino group.
A) The top chromatogram show the reference amino oligo O: 5′-GAC CTG TCG AGC ATC CAG CTT CAT GGC TGA GTC CAC AAT GZ. Z contain the modified nucleobase with an aminogroup, incorporated using the commercially available amino modifier C6 dT phosphoramidite (10-1039-90 from Glen research).
B) The middle chromatogram show the streptavidine purified amino oligo O after partial transfer of a acetyl group from oligo L.
C) The bottom chromatogram show the streptavidine purified amino oligo O after the complete transfer of the more lipophilic 3-tertbutoxycarbonylamino-butanyl. The following gradient was used in the obtainment of the chromatograms: 0-3 minutes 100% A then 15% A and 85% B from 3-10 minutes.

The experiment where the template oligo was omitted showed no non-templated product formation. The results indicate that the efficiency of the templated synthesis was 80-100%. The reason for less than 100% efficiency was probably due to hydrolytic cleavage of the functional entity.

### EXAMPLE 8

### Simultaneous Transfer of Two Functional Entities

The following oligo containing a nucleobase modified with a carboxylic acid moiety, was synthesised using the conventional phosphoramidite approach:

H: 5′-GAC CTG TCG AGC ATC CAG CTT CAT GGG AAT TCC
TCG TCC ACA ATG XT

X was incorporated using the commercially available carboxy-dT phosphoramidite (10-1035-90 from Glen research).

The modified oligo was provided with a trisamine scaffold according to the scheme:

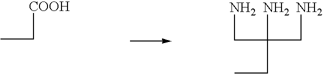

Procedure: The modified oligo (1 nmol) was mixed with water (100 uL), hepes buffer (40 uL of a 200 mM, pH=7.5), NHS (20 uL of a 100 mM solution), EDC (20 uL of a freshly prepared 1 M solution) and the tetraamine tetrakis(aminomethyl)methane tetrahydrochloride (20 uL of a 100 mM solution). The reaction mixture was left o/n at room temperature. The volume was reduced to 60 uL by evaporation in vacuo. The pure oligo was obtained by addition of NH₃ conc. (20 uL) followed by HPLC purification. It was possible to isolate a peak after approximately 6 min using the following gradient: 0-3 minutes 100% A then 15% A and 85% B from 3-10 minutes then 100% B from 10-15 minutes then 100% A from 15-20 minutes. A=2% acetonitrile in 10 mM TEAA and B=80% acetonitrile in 10 mM TEAA.

The following oligos containing a nucleobase modified with a S-triphenylmethyl protected thio moiety, was synthesised using the conventional phosphoramidite approach:

K: 5′-WCA TTG ACC TGT CTG CCB TGT CAG TCG GTA CTG
TGG TAA CGC GGA TCG ACC T
L: 5′-WCA TTG ACC TGA ACC ATG BTA AGC TGC CTG TCA
GTC GGT ACT ACG ACT ACG TTC AGG CAA GA

W was incorporated using the commercially available thiol modifier phosphoramidite (10-1926-90 from Glen research). B is an internal biotin incorporated using the commercially available phosphoramidite (10-1953-95 from Glen research).

To make an SH group available for further reaction, the S-triphenylmethyl protected thio oligo (10 nmol) was evaporated in vacuo and resuspended in TEAA buffer (200 uL of a 0.1M solution, pH=6.4). AgNO₃ (30 uL of a 1 M solution) was added and the mixture was left at room temperature for 1-2 hours. DTT (46 uL of a 1 M solution) was added and left for 5-10 minutes. The reaction mixture was spun down (20.000 G for 20 minutes) and the supernatant was collected. The solid was extracted with additional TEAA buffer (100 ul of a 0.1.M solution, pH=6.4). The pure thio oligo was obtained by conventional EtOH-precipitation.

The K and L oligo was subsequently reacted with the compound 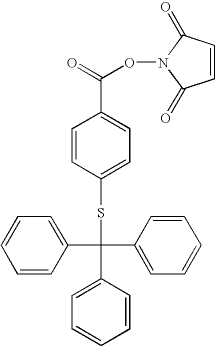
forming a building block capable of transferring the lipophilic S-Trityl-4-mercaptobenzoyl group to a recipient nucleophilic group.

The transfer reaction is schematically represented below:

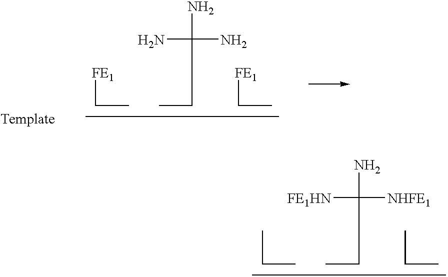

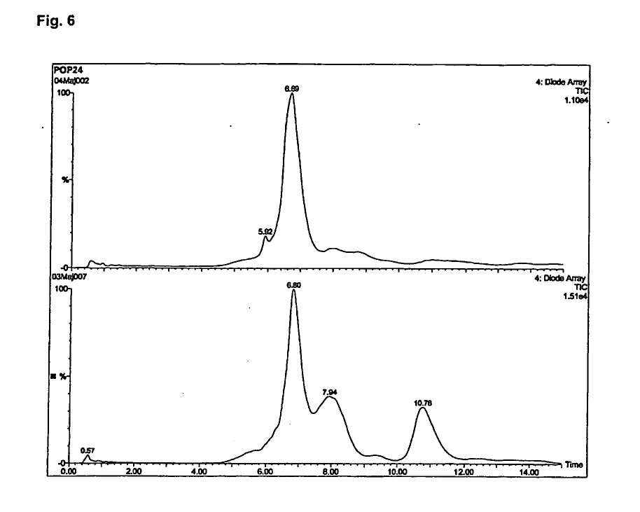

The template oligo 5′-BTCTTGCCTGAACGTAGTCGTAGGTCGATCCGCGTTACCAGAGCTGGATGCTC GACAGGTCCCGATGCAATCCAGAGGTCG (1 nmol) was mixed with the two thio oligos (K and L) loaded with the same functional entity (S-Trityl-4-mercaptobenzoyl; 1 nmol) and the trisamine oligo H (1 nmol) in hepes-buffer (20 uL of a 100 mM hepes and 1 M NaCl solution, pH=7.5) and water (added to a final volume of 100 uL). The oligos were annealed to the template by heating to 50° C. and cooled (−2° C./30 second) to 30° C. The mixture was then left o/n at a fluctuating temperature (10° C. for 1 second then 35° C. for 1 second). The oligo complex was attached to streptavidine by addition of streptavidine beads (100 uL, prewashed with 2×1 mL 100 mM hepes buffer and 1M NaCl, pH=7.5). The beads were washed with hepes buffer (1 mL). The trisamine scaffold oligo H was separated from the streptavidine bound complex by addition of water (200 uL) followed by heating to 70° C. The water was transferred and evaporated in vacuo, resuspended in TEAA buffer (45 uL of a 0.1 M solution) and product formation analysed by HPLC (see FIG. 6).

The HPLC chromatogram shows the transfer of two functional entities to a scaffold oligo with three amino groups.
A) The top chromatogram shows the reference scaffold oligo H.
B) The bottom chromatogram show the streptavidine purified scaffold oligo H after the partial transfer of one (peak at 7.94 minutes) and two (peak at 10.76 minutes) identical functional entities (S-Trityl-4-mercaptobenzoyl). The following gradient was used: 0-3 minutes 100% A, then 15% A, and 85% B from 3-10 minutes then 100% B from 10-15 minutes. A=2% acetonitrile in 10 mM TEAA and B=80% acetonitrile in 10 mM TEAA.

Due to the lipophilic nature of the functional entities a longer retention time, in the HPLC chromatogram of the scaffolded molecule with two functional entities compared to one functional entity, was observed. The efficiency of the templated synthesis of a scaffolded molecule with the two identical functional entities was about 25% (peak at 10.76 minutes in FIG. 6).

### Model Example 1

General route to the formation of acylating building blocks and the use of these:

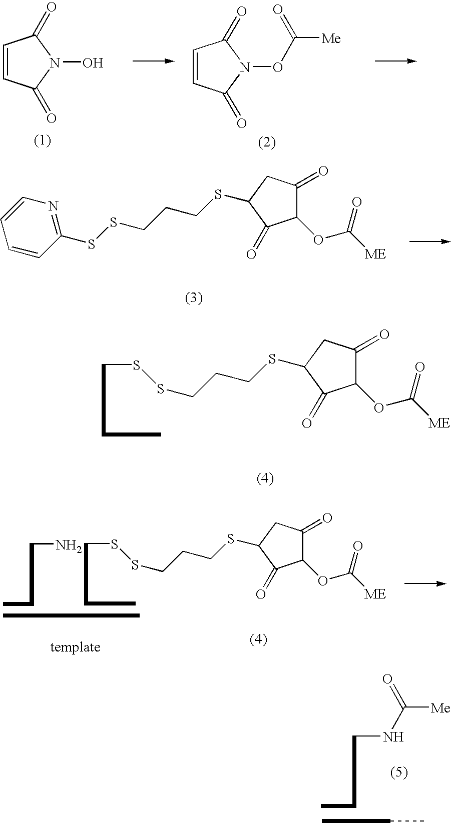

N-hydroxymaleimide (1) may be acylated by the use of an acylchloride e.g. acetyl-chloride or alternatively acylated in e.g. THF by the use of dicyclohexylcarbodiimide or diisopropylcarbodiimide and acid e.g. acetic acid. The intermediate may be subjected to Michael addition by the use of excess 1,3-propanedithiol, followed by reaction with either 4,4′-dipyridyl disulfide or 2,2′-dipyridyl disulfide. This intermediate (3) may then be loaded onto an oligonucleotide carrying a thiol handle to generate the building block (4). The reaction of this building block with an amine carrying scaffold is conducted as follows:

The template oligonucleotide (1 nmol) is mixed with a thio oligonucleotide building block e.g. (4) (1 nmol) and an amino-oligonucleotide scaffold (1 nmol) in hepes-buffer (20 μL of a 100 mM hepes and 1 M NaCl solution, pH=7.5) and water (39 uL). The oligonucleotides are annealed to the template by heating to 50° C. and cooling (2° C./second) to 30° C. The mixture is then left o/n at a fluctuating temperature (10° C. for 1 second then 35° C. for 1 second), to yield template bound (5).

The above examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full content of this document, including the examples shown above and the references to the scientific a patent literature cited herein. It should further be appreciated that the contents of those cited references are incorporated herein by reference to help illustrate the state of the art. The examples above contain important additional information that can be adapted to the practice of this invention in its various embodiments and the equivalents thereof.

Abbreviations

| | |
|---|---|
| DCC | N,N′-Dicyclohexylcarbodiimide |
| DhbtOH | 3,4-dihydro-3-hydroxy-4-oxo-1,2,3-benzotriazine |
| DIC | Diisopropylcarbodiimide |
| DIEA | Diethylisopropylamin |
| DMAP | 4-Dimethylaminopyridine |
| DNA | Deoxyribosenucleic Acid |
| EDC | 1-Ethyl-3-(3′-dimethylaminopropyl)carbodiimide.HCl |
| HATU | 2-(1H-7-Azabenzotriazole-1-yl)-1,1,3,3-tetramethyluronium |
| | hexafluorophosphate |
| HBTU | 2-(1H-Benzotriazole-1-yl)-1,1,3,3-tetramethyluronium |
| | hexafluorophosphate |
| HOAt | N-Hydroxy-7-azabenzotriazole |
| HOBt | N-Hydroxybenzotriazole |
| LNA | Locked Nucleic Acid |
| NHS | N-hydroxysuccinimid |
| OTf | Trifluoromethylsulfonate |
| OTs | Toluenesulfonate |
| PNA | Peptide Nucleic Acid |
| PyBoP | Benzotriazole-1-yl-oxy-tris-pyrrolidino- |
| | phosphonium hexafluorophosphate |
| PyBroP | Bromo-tris-pyrrolidino-phosphonium hexafluorophosphate |
| RNA | Ribonucleic acid |
| TBTU | 2-(1H-Benzotriazole-1-yl)-1,1,3,3- |
| | tetramethyluronium tetrafluoroborate |
| TEA | Triethylamine |
| RP-HPLC | Reverse Phase High Performance Liquid Chromatography |
| TBDMS-Cl | Tert-Butyldimethylsilylchloride |
| 5-Iodo-dU | 5-iodo-deoxyriboseuracil |
| TLC | Thin layer chromatography |
| (Boc)₂O | Boc anhydride, di-tert-butyl dicarbonate |
| TBAF | Tetrabutylammonium fluoride |
| SPDP | Succinimidyl-propyl-2-dithiopyridyl |
| CTAB | Cetylammoniumbromide |

1. A building block of the general formula 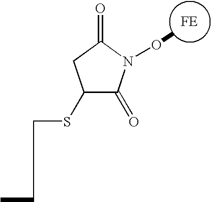
capable of transferring a functional entity (FE) to a recipient reactive group, wherein
the lower horizontal line is a Complementing Element identifying the functional entity and the vertical line between the complementing element and the S atom is a Spacer.

2. The building block of claim 1, wherein the spacer is a valence bond, C₁-C₆ alkylene-A-, C₁-C₆ alkenylene-A-, C₂-C₆ alkynylene-A-, or 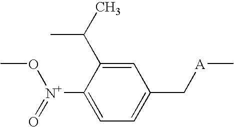
said spacer optionally being connected through A to a moiety selected from 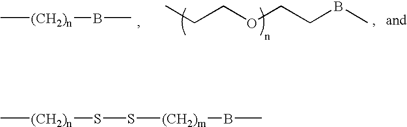
where A is a valence bond, —C(O)NR¹—, —NR¹—, —O—, —S—, or —C(O)—O—; B is a valence bond, —O—, —S—, —NR¹— or —C(O)NR¹— and connects to the S atom of the carrier; R¹ is selected independently from H, C₁-C₆ alkyl, C₃-C₇cycloalkyl, C₁-C₆ alkylene-aryl, or aryl substituted with 0-5 halogen atoms selected from —F, —Cl, —Br and —I; and n and m independently are integers ranging from 1 to 10.

3. The compound according to claim 1, wherein the Spacer is C₁-C₆ alkylene-A-, C₁-C₆ alkenylene-A-, C₂-C₆ alkynylene-A-, or 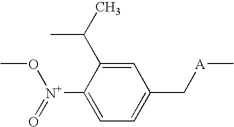
said spacer optionally being connected through A to a moiety selected from 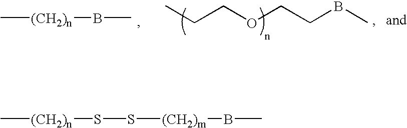
where A is —C(O)NR¹—, or —S—; B is —S—, —NR¹— or —C(O)NR¹— and connects to S-C-connecting group; R¹ is selected independently from H, C₁-C₆ alkyl, C₁-C₆ alkylene-aryl, or aryl; and n and m independently are integers ranging from 1 to 6.

4. The compound according to claim 1, wherein Spacer is -A-, a group C₁-C₆ alkylene-A-, C₂-C₆ alkenylene-A-, or C₂-C₆ alkynylene-A- optionally substituted with 1 to 3 hydroxy groups, or 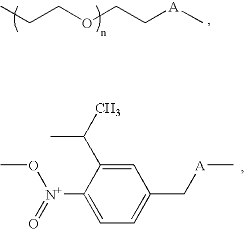
said spacer being connected through A to a linker selected from 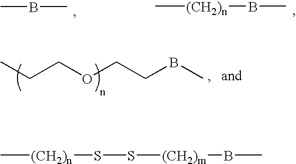
where A is a valence bond, —NR²—, —C(O)NR²—, —NR—C(O)—, —O—, —S—, —C(O)—O— or —OP(═O)(O⁻)—O—; B is a valence bond, —O—, —S—, —NR²—, —C(O)— or —C(O)NR²— and connects to S-C-connecting group; R is selected independently from H, C₁-C₆ alkyl, C₃-C₇ cycloalkyl, aryl, C₁-C₆ alkylene-aryl, 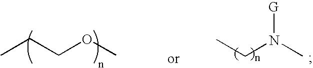
G is H or C₁-C₆ alkyl; and n and m independently are integers ranging from 1 to 10.

5. A compound according to claim 4, wherein the spacer is C₂-C₆ alkenylene-A, said spacer being connected through A to a moiety selected from 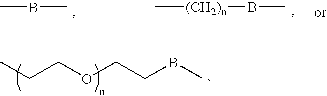
where A is a valence bond, —C(O)NR²—, —NR—C(O)—, —S—, —C(O)—O— or —OP(═O)(O⁻)—O—; B is a valence bond, —S—, —NR²—, or —C(O)— and connects to S-C-connecting group; n and m independently are integers ranging from 1 to 10 and R² is selected independently from H, 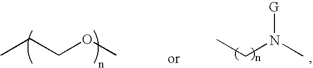
wherein G is H or C₁-C₆ alkyl; and the spacer is connected to the complementing element through a nucleobase.

6. A compound according to claim 4, wherein the spacer is -A-, 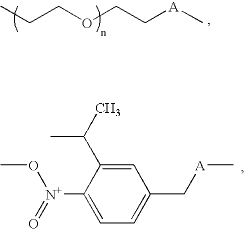
said spacer being connected through A to a moiety selected from 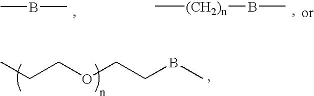
where A is a valence bond, —NR²—C(O)—, —O—, or —S—; B is a valence bond, —S—, —NR²—, or —C(O)— and connects to S-C-connecting group;
n and m independently are integers ranging from 1 to 10 and R² is selected independently from H, 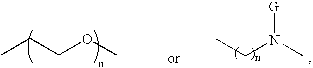
wherein G is H or C₁-C₆ alkyl; and the spacer is connected to the complementing element via a phosphorus group.

7. A compound according to claim 6, wherein the phosphorus group is a phosphate or thiophosphate group attached to a 3′ or 5′ end of a complementing element.

8. The building block according to claim 1, wherein FE is 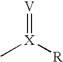
where
X=—C—, —S—, —P—, —S(O)—, or —P(O)—,
V=O, S, NH, or N-C₁-C₆ alkyl, and
R is H or selected among the group consisting of a C₁-C₆ alkyl, C₂-C₆ alkenyl, C₂-C₆ alkynyl, C₄-C₈ alkadienyl, C₃-C₇cycloalkyl, C₃-C₇cycloheteroalkyl, aryl, and heteroaryl, said group being substituted with 0-3 R⁴, 0-3 R⁵ and 0-3 R⁹ or C₁-C₃alkylene-NR⁴₂, C₁-C₃alkylene-NR⁴C (O)R⁸, C₁-C₃alkylene-NR⁴C(O)OR⁸, C₁-C₂alkylene-O—NR⁴₂, C₁-C₂alkylene-O—NR⁴C(O)R⁸, C₁-C₂alkylene-O—NR⁴C(O)OR⁸ substituted with 0-3 R⁹,
where R⁴ is H or selected independently among the group consisting of C₁-C₆ alkyl, C₂-C₆ alkenyl, C₂-C₆ alkynyl, C₃-C₇cycloalkyl, C₃-C₇cycloheteroalkyl, aryl, heteroaryl, said group being substituted with 0-3 R⁹ and
R⁵ is selected independently from —N₃, —CNO, —C(NOH)NH₂, —NHOH, —NHNHR⁶, —C(O)R⁶, —SnR⁶₃, —B(OR⁶)₂, —P(O)(OR⁶)₂ or the group consisting of C₂-C₆ alkenyl, C₂-C₆ alkynyl, C₄-C₈alkadienyl said group being substituted with 0-2 R⁷,
where R⁶ is selected independently from H, C₁-C₆ alkyl, C₃-C₇cycloalkyl, aryl or C₁-C₆ alkylene-aryl substituted with 0-5 halogen atoms selected from —F, —Cl, —Br, and —I; and R⁷ is independently selected from —NO₂, —COOR⁶, —COR⁶, —CN, —OSiR⁶₃, —OR⁶ and —NR⁶₂,
R⁸ is H, C₁-C₆ alkyl, C₂-C₆ alkenyl, C₂-C₆ alkynyl, C₃-C₇cycloalkyl, aryl or C₁-C₆ alkylene-aryl substituted with 0-3 substituents independently selected from —F, —Cl, —NO₂, —R³, —OR³, —SiR³₃
R⁹ is ═O, —F, —Cl, —Br, —I, —CN, —NO₂, —OR⁶, —NR⁶₂, —NR⁶—C(O)R⁸, —NR⁶—C(O) OR⁸, —SR⁶, —S(O)R⁶, —S(O)₂R⁶, —COOR⁶, —C(O)NR⁶₂ and —S(O)₂NR⁶₂.

9. A compound according to claim 8, wherein R is H or selected among the group consisting of a C₁-C₆ alkyl, C₂-C₆ alkenyl, C₂-C₆ alkynyl, C₄-C₆ alkadienyl, C₃-C₇ cycloalkyl, C₃-C₇ cycloheteroalkyl, aryl, and heteroaryl, said group being substituted with 0-3 R⁵ and 0-3 R⁹, or selected among the group consisting of C₁-C₃alkylene-NR⁴₂, C₁-C₃alkylene-NR⁴C(O)R⁸, C₁-C₃alkylene-NR⁴C(O)OR⁸, C₁-C₂ alkylene-O—NR⁴₂, C₁-C₂ alkylene-O—NR⁴C(O)R⁸, and C₁-C₂ alkylene-O—NR⁴C(O)OR⁸ substituted with 0-3 R⁹.

10. A compound according to claim 8, wherein R is H or selected among the group consisting of C₁-C₆ alkyl, C₂-C₆ alkenyl, C₂-C₆ alkynyl, C₄-C₈ alkadienyl, C₃-C₇ cycloalkyl, C₃-C₇ cycloheteroalkyl, aryl, and heteroaryl, said group being substituted with 0-3 R⁵ and 0-3 R⁹.

11. A compound according to claim 8, wherein R is selected among the group consisting of C₁-C₃alkylene-NR⁴₂, C₁-C₃alkylene-NR⁴C(O)R⁸, C₁-C₃alkylene-NR⁴C(O)OR⁸, C₁-C₂ alkylene-O—NR⁴₂, C₁-C₂ alkylene-O—NR⁴C(O)R⁸, and C₁-C₂ alkylene-O—NR⁴C(O)OR⁸ substituted with 0-3 R⁹.

12. A compound according to claim 1, wherein X═C and V═O or S.

13. A compound according to claim 1, wherein X═C and V═O.

14. A compound according to claim 1, wherein complementing element is a nucleic acid.

15. A compound according to claim 1, wherein Complementing element is a sequence of nucleotides selected from the group of DNA, RNA, LNA PNA, or morpholino derivatives.

16. A library of compounds according to claim 1, wherein each different member of the library comprises a complementing element having a unique sequence of nucleotides, which identifies the functional entity.

17. A method for transferring a functional entity to a recipient reactive group, comprising the steps of
providing one or more building blocks according to claim 1,
contacting the one or more building blocks with a corresponding encoding element associated with a recipient reactive group under conditions which allow for a recognition between the one or more complementing elements and the coding elements, said contacting being performed prior to, simultaneously with, or subsequent to a transfer of the functional entity to the recipient reactive group.

18. The method according to claim 17, wherein the coding element comprises one or more coding sequences comprised of 1 to 50 nucleotides and the one or more complementing elements comprises a sequence of nucleotides complementary to one or more of the coding sequences.

19. The method of claim 17, wherein the recipient reactive group is an amine group, which may be part of a chemical scaffold, and the linkage between the functional entity and the scaffold is of the general chemical structure:
Scaffold-NH—X(═V)—R
In which
X=—C—, —S—, —P—, —S(O)—, —P(O)—, and
V=O, S, NH, N-C₁-C₆ alkyl.

20. The method according to claim 19, wherein X is C and V is O.

21. A process for preparing a building block according to claim 1, comprising the step of

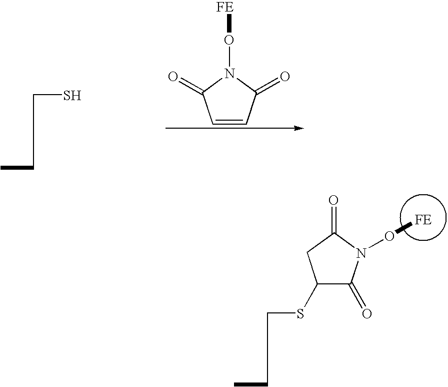

22. A process for preparing a building block according to claim 1, comprising the steps of 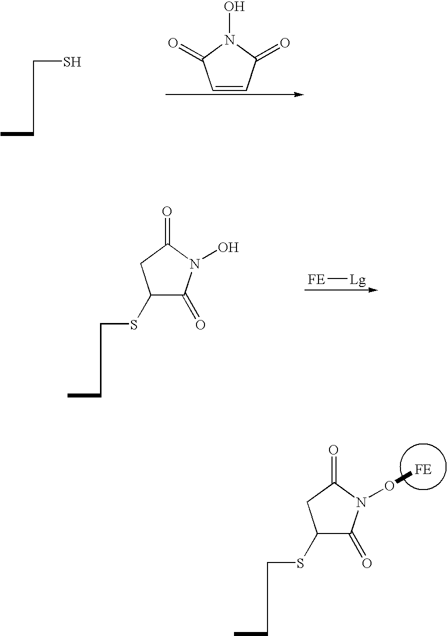
where Lg is a leaving group.

23. A process according to claim 18, wherein the leaving group is selected from 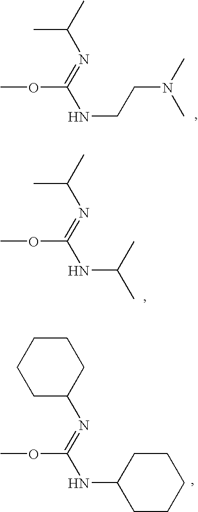
Cl, Br.
